(19) United States
(12) Patent Application Publication
(10) Pub. No.: US 2006/0140988 A1
(43) Pub. Date: Jun. 29, 2006

(54) Visco-supplement composition and methods

(76) Inventors: Guohua Chen, Sunnyvale, CA (US); Edwin Chan, San Francisco, CA (US); Joel Rosenblatt, Portstown, PA (US)

Correspondence Address: WOODCOCK WASHBURN LLP, ONE LIBERTY PLACE, 46TH FLOOR, PHILADELPHIA, PA, 19103, US

(21) Appl. No.: 11/305,939

(22) Filed: Dec. 19, 2005

Related U.S. Application Data
(60) Provisional application No. 60/638,535, filed on Dec. 23, 2004.

(51) Int. Cl. A61K 31/728; A61K 9/00; A61K 9/14

(52) U.S. Cl. 424/400; 424/486; 514/54

(57) ABSTRACT

The present invention relates to methods and depot emulsion compositions for delivery of visco-supplements.

### CROSS REFERENCE

This application claims benefit to U.S. Provisional Application Serial No. 60/638,535, filed Dec. 23, 2004, the disclosure of which is incorporated herein by reference in its entirety.

### FIELD

The present invention relates to methods and compositions for delivery of visco-supplements.

### BACKGROUND

Osteoarthritis is a degenerative joint disease characterized by progressive degradation of the cartilage matrix. This results in pain and stiffness, with eventual loss of function. One approach to treating osteoarthritis is to place a visco-supplement in the joint to relieve pain. For example, one such visco-supplement is hyaluronic acid or a salt thereof. Solutions of highly purified, high molecular weight (2.4-3.6 million Daltons) or lightly crosslinked sodium hyaluronate in phosphate-buffered saline has been used as a visco-supplement to relieve pain caused by osteoarthritis, specifically in the knee joint. It is believed that hyaluronate provides lubrication at the joint allowing greater mobility and flexibility of the treated location. It may also function to irrigate the articular, or joint, space.

However, aqueous solutions of hyaluronate are quickly absorbed by the body. Some studies indicate that injections of hyaluronate into the joint space remain there for only a few days. This requires frequent injections, which are uncomfortable and time-consuming.

Therefore, what is needed are improved compositions and methods for delivery of visco-supplements. The present invention is directed to these, as well as other important ends.

### SUMMARY

The present invention describes compositions comprising a biodegradable polymer, a solvent, a visco-supplement such as hyaluronic acid or a salt thereof, and a surfactant. In some embodiments, the composition is an injectable depot emulsion composition for the sustained delivery of hyaluronic acid or a salt thereof.

The present invention also describes methods of administering a visco-supplement, comprising forming an emulsion containing the visco-supplement, and injecting said emulsion into a patient in need thereof.

### DETAILED DESCRIPTION

In one embodiment, the present invention includes a composition comprising a biodegradable polymer, a solvent, a visco-supplement, for example, hyaluronic acid or a salt thereof, and a surfactant.

The term visco-supplement is intended to comprise any known compounds administered to the articular space, particularly those for providing lubrication. In one embodiment, the visco-supplement is hyaluronic acid or a salt thereof. Hyaluronic acid is a polysaccharide composed of repeat disaccharide units of N-acetylglucosamine and glucuronic acid. Hyaluronic acid has high viscoelasticity and lubricity, and is found in many animals. It can be isolated from rooster combs, or expressed by bacteria and purified. In one embodiment, the salt is sodium hyaluronate. Sodium hyaluronate is commercially available also, and is an ingredient in many visco-supplements, such as SYNVISC® available from Genzyme, HYALGAN® available from Fidia, and ARTHREASE® available from Bio-Technology General.

In one embodiment, the hyaluronic acid or a salt thereof is present in a range from about 0.1 wt. % to about 50 wt. % of the composition.

In one embodiment, the hyaluronic acid is in a dry form. In one embodiment, the dry form is about 0.1 -50 wt. % of the composition, preferably about 1-30 wt. % of the composition.

In yet another embodiment, the hyaluronic acid is an aqueous solution. In one embodiment of the aqueous solution of hyaluronic acid, the hyaluronic acid concentration is about 0.5-5 wt. %, while the aqueous solution of hyaluronic acid in the composition is about 30-50 wt. %.

It is understood that the composition may be formed in a variety of ways. For example, in one embodiment, the biodegradable polymer and the solvent comprise a depot vehicle. In injectable embodiments, it is important to have a stable homogenous emulsion in order to easily and consistently inject the mixture via a small diameter needle. The presence of a surfactant in the depot vehicle can induce homogeneous emulsions and easier injection. In such an embodiment, the depot vehicle may further comprise the surfactant.

In one embodiment, the depot vehicle employs ALZAMER depot technology available from ALZA. Such a vehicle provides sustained release as the polymer in the depot vehicle can form a matrix system for controlled delivery of hyaluronate.

In one embodiment, the biodegradable polymer is selected from the group consisting of polylactides, polyglycolides, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), and poly(amino acids), and copolymers, terpolymers and mixtures thereof.

The biodegradable polymer may be present in a range from about 15 wt % to about 60 wt % of the composition.

In some embodiments, the biodegradable polymer is a lactic acid-containing polymer. The lactic acid may be present in a range from about 1 wt. % to about 100 wt. % of the polymer. In some embodiments, the lactic acid is present in a range from about 25 wt. % to about 75 wt. % of the polymer.

In some embodiments, the biodegradable polymer is a copolymer of lactic acid and glycolic acid. When lactic acid is present, the glycolic acid may be present in a range from about 35 wt. % to about 65 wt. % of the polymer. In other embodiments, the lactic acid is present in a range from about 45 wt. % to about 99 wt. % of the polymer.

In some embodiments, the biodegradable polymer is a terpolymer of lactic acid, glycolic acid, and poly ε-caprolactone. In some embodiments, the biodegradable polymer is a terpolymer of 5 wt. % lactic acid, 55 wt. % glycolic acid, and 40 wt. % poly ε-caprolactone.

In one embodiment, the solvent is selected from the group consisting of aromatic alcohols, lower alkyl esters of aryl acids, lower aralkyl esters of aryl acids, aryl ketones, aralkyl ketones, lower alkyl ketones, and lower alkyl esters of citric acid, and combinations thereof. In one embodiment, the solvent is ethyl oleate, benzyl benzoate, ethyl benzoate, lauryl lactate, benzyl alcohol, lauryl alcohol, glycofurol, ethanol, tocopherol, polyethylene glycol, triacetin, a triglyceride, an alkyltriglyceride, a diglyceride, sesame oil, peanut oil, castor oil, olive oil, cottonseed oil, perfluorocarbon, N-methyl-pyrrolidone, DMSO, glycerol, oleic acid, glycofurol, lauryl lactate, perfluorocarbon, propylene carbonate, or mixtures thereof. In one embodiment, the solvent is methyl benzoate, ethyl benzoate, n-propyl benzoate, isopropyl benzoate, butyl benzoate, isobutyl benzoate, sec-butyl benzoate, tert-butyl benzoate, isoamyl benzoate, or benzyl benzoate. In one embodiment, the solvent is benzyl benzoate. In one embodiment, the solvent is benzyl alcohol. And in another embodiment, the solvent is benzyl benzoate and benzyl alcohol.

In one embodiment, the solvent is present in a range from about 15 wt % to about 60 wt % of the composition.

In one embodiment, the surfactant is an ionic surfactant, nonionic surfactant, or a polymeric surfactant. Examples of surfactants include ALKANOL® 189-S, ALKANOL® XC, Allyl alcohol 1,2-butoxylate-block-ethoxylate, ammonium sulfate end-capped solution, 80 wt. % in propylene glycol, 1-Decanesulfonic acid sodium salt, 98%, 4-(2,3-Dihydroxypropyl) 2-(2-methylene-4,4-dimethylpentyl)succinate potassium salt solution, 40 wt. % in water, N,N-Dimethyl-N-[3-(sulfooxy)propyl]-1-decanaminium hydroxide inner salt, N,N-Dimethyl-N-[3-(sulfooxy)propyl]-1-nonanaminium hydroxide inner salt, Dioctyl sulfosuccinate sodium salt, 96%, N-Ethyl-N-[(heptadecafluorooctyl)sulfonyl]glycine potassium salt solution, 42 wt. % in water/2-butoxyethanol, Glycolic acid ethoxylate 4-tert-butylphenyl ether, Average MN˜380, Glycolic acid ethoxylate lauryl ether, Average MN˜360, Glycolic acid ethoxylate lauryl ether, Average MN˜460, Glycolic acid ethoxylate lauryl ether, Average MN˜690, Glycolic acid ethoxylate 4-nonylphenyl ether, Average MN˜600, Glycolic acid ethoxylate oleyl ether, Average MN˜410, Glycolic acid ethoxylate oleyl ether, Average MN˜540, Glycolic acid ethoxylate oleyl ether, Average MN˜700, [3-((((Heptadecafluorooctyl)sulfonyl)amino)propy)]trimethylammonium iodide solution, 42 wt. % in 2-propanol/water, Poly(ethylene glycol) 4-nonylphenyl 3-sulfopropyl ether potassium salt, Sodium dodecylbenzenesulfonate, Technical Grade, Sodium dodecyl sulfate, 70%, Sodium dodecyl sulfate, 98%, ZONYL® 7950, ZONYL®FSA fluorosurfactant, 25 wt. % Li carboxylate salt in water: isopropanol (37.5:37.5)., ZONYL® FSE fluorosurfactant, 14 wt. % in water: ethylene glycol (62:24), ZONYL® FSP fluorosurfactant, ZONYL®UR fluorosurfactant, ADOGEN® 464, ALKANOL® 6112, Allyl alcohol 1,2-butoxylate-block-ethoxylate, Allyl alcohol 1,2-butoxylate-block-ethoxylate, BRIJ®30, Average MN˜362, BRIJ®35, Average MN˜1,198, BRIJ®52, Average MN˜330, BRIJ®56, Average MN˜683, BRIJ®58, Average MN—1,124, BRIJ®72, Average MN˜359, BRIJ®76, Average MN˜711, BRIJ®78, Average MN˜1,152, BRIJ®92, Average MN˜357, BRIJ®97, Average MN˜709, BRIJ®98, Average MN˜1,150, BRIJ® 700, Average MN˜4,670, 2,5-Dimethyl-3-hexyne-2,5-diol, 98%, Ethylenediamine tetrakis(ethoxylate-block-propoxylate) tetrol, Average MN˜7,200, Ethylenediamine tetrakis(ethoxylate-block-propoxylate) tetrol, Average MN˜8,000, Ethylenediamine tetrakis(propoxylate-block-ethoxylate) tetrol, Average MN˜3,600, Ethylenediamine tetrakis(propoxylate-block-ethoxylate) tetrol, Average MN˜15,000, IGEPAL® CA-210, Average MN˜294, IGEPAL® CA-520, Average MN˜427, IGEPAL® CA-720, Average MN˜735, IGEPAL® CO-210, Average MN˜308, IGEPAL® CO-520, IGEPAL® CO-630, Average MN˜617, IGEPAL® CO-720, Average MN˜749, IGEPAL® CO-890, Average MN˜1,982, IGEPAL® CO-990, Average MN˜4,626, IGEPAL® DM-970, MERPOL® DA surfactant, 60 wt. % in water/isobutanol (ca. 50:50), MERPOL® HCS surfactant, MERPOL® LFH surfactant, MERPOL® OJ surfactant, MERPOL® SE surfactant, MERPOL® SH surfactant, MERPOL®A surfactant, 8-Methyl-1-nonanol propoxylate-block-ethoxylate, Poly(acrylic acid) partial sodium salt, particle size 1000 μm (99%), Poly(acrylic acid) partial sodium salt solution, Average MW˜2,000 by GPC, 60 wt. % in water, Poly[dimethylsiloxane-co-methyl(3-hydroxypropyl)siloxane]-g raft-poly(ethylene/propylene glycol), Polyethylene-block-poly(ethylene glycol), Average MN˜1,400, Polyethylene-block-poly(ethylene glycol), Average MN˜920, Polyethylene-block-poly(ethylene glycol), Average MN˜875, Polyethylene-block-poly(ethylene glycol), Average MN˜575, Poly(ethylene glycol) n-alkyl 3-sulfopropyl ether potassium salt, Poly(ethylene glycol)-block-poly(propylene glycol)-block-poly(ethylene glycol), Average MN˜1,100, Poly(ethylene glycol)-block-poly(propylene glycol)-block-poly(ethylene glycol), Average MN˜1,900, Poly(ethylene glycol)-block-poly(propylene glycol)-block-poly(ethylene glycol), Average MN˜2,000, Poly(ethylene glycol)-block-poly(propylene glycol)-block-poly(ethylene glycol), Average MN˜2,800, Poly(ethylene glycol)-block-poly(propylene glycol)-block-poly(ethylene glycol), Average MN˜2,800, Poly(ethylene glycol)-block-poly(propylene glycol)-block-poly(ethylene glycol), Average MN˜2,900, Poly(ethylene glycol)-block-poly(propylene glycol)-block-poly(ethylene glycol), Average MN˜4,400, Poly(ethylene glycol)-block-poly(propylene glycol)-block-poly(ethylene glycol), Average MN˜5,800, Poly(ethylene glycol)-block-poly(propylene glycol)-block-poly(ethylene glycol), Average MN˜8,400, Poly(ethylene glycol) 2-[ethyl[(heptadecafluorooctyl)sulfonyl]amino]ethyl ether, Poly(ethylene glycol) 2-[ethyl[(heptadecafluorooctyl)sulfonyl]amino]ethyl methyl ether, Poly(ethylene glycol) myristyl tallow ether, Average MN˜3,000, Poly(hexafluoropropylene oxide) monocarboxylic acid, chloro terminated, Average MN˜500, Polyoxyethylene sorbitan tetraoleate, Polyoxyethylene sorbitol hexaoleate, Polyoxyethylene(6) tridecyl ether, Mixture of C11 to C14 iso-alkyl ethers with C13 iso-alkyl predominating., Polyoxyethylene(12) tridecyl ether, Mixture of C11 to C14 iso-alkyl ethers with C13iso-alkyl predominating., Polyoxyethylene(18) tridecyl ether, Mixture of C11 to C14 iso-alkyl ethers with C13 iso-alkyl predominating., Poly(propylene glycol)-block-poly(ethylene glycol)-block-poly(propylene glycol), Average MN˜2,000, Poly(propylene glycol)-block-poly(ethylene glycol)-block-poly(propylene glycol), Average MN˜2,700, Poly(propylene glycol)-block-poly(ethylene glycol)-block-poly(propylene glycol), Average MN˜3,300, Sorbitan monolaurate, Sorbitan monooleate, Sorbitan monopalmitate, Sorbitan monostearate, Sorbitan sesquioleate, Sorbitan trioleate, TERGITOL® NP-9, 2,4,7,9-Tetramethyl-5-decyne-4,7-diol ethoxylate, Average MN˜380, Average MW˜395, 2,4,7,9-Tetramethyl-5-decyne-4,7-diol ethoxylate, Average MN˜670, Average MW˜700, 2,4,7,9-Tetramethyl-5-decyne-4,7-diol ethoxylate, Average MN˜1,200, Average MW˜1,250, 2,4,7,9-Tetramethyl-5-decyne-4,7-diol, mixture of (±) and meso, 98%, TRITON® X-100, TRITON® X-100, reduced, TRITON® N-101, reduced, TRITON® X-114, TRITON® X-114, reduced, 99+%, TRITON® X-114, reduced, TRITON® X-405, reduced, TRITON® X-405 solution, 70 wt. % in water, TRITON® SP-135, TRITON® SP-190, TWEEN® 20, Average MN˜1,228, TWEEN®20 solution, 72 wt. % in water, TWEEN® 40, Average MN˜1,284, TWEEN® 60, Average MN˜1,312, TWEEN® 80, Average MN˜1,310, TWEEN® 85, Average MN—1,839, PLURONIC® F68, PLURONIC® F127, PLURONIC® L61, PLURONIC® L81, PLURONIC® L92, PLURONIC® L121 etc, TWEEN 20, TWEEN 80, CREMOPHOR® EL 35, CREMOPHOR® EL 40, CREMOPHOR® EL 60, ZONYL® FSN, ZONYL® FSN-100, ZONYL® FSO, and ZONYL® FSO-100.

In one embodiment, the surfactant is present in a range from about 0.01 wt % to about 5 wt % of the composition. In some embodiments, the surfactant is present in a range from about 0.04 wt. % to about 2.0 wt. % of the composition.

In one embodiment, the surfactant is polymeric surfactant. In one embodiment, the polymeric surfactant is present in a range from about 0.01 wt % to about 5 wt % of the composition. In some embodiments, the surfactant is present in a range from about 0.04 wt. % to about 2.0 wt. % of the composition.

In some embodiments, the surfactant is a polyoxyethylene sorbitan-containing composition or a block copolymer of propylene oxide and ethylene oxide, a block copolymer derived from the addition of ethylene oxide and propylene oxide to ethylenediamine, polyethelene glycol, or polyethylene oxide. In one embodiment, the surfactant is TWEEN 20 (polyoxyethylene sorbitan monolaureate) or TWEEN 80 (polyoxyethylene sorbitan monooleat).

In some embodiments, the surfactant is a block copolymer of propylene oxide and ethylene oxide is of a formula HO-(ethylene oxide)ₓ-(propylene oxide)_{y}-(ethylene oxide)_{x′}-H. In one embodiment, x is in a range from about 2 to about 150, y is in a range from about 20 to about 70, and x′ is in a range from about 2 to about 150. In one embodiment, x is about 79, y is about 28, and x′ is about 79. In one embodiment, the surfactant is PLURONIC F68 surfactant.

The present invention provides an injectable depot emulsion composition for the sustained delivery of hyaluronic acid or a salt thereof comprising the above described compositions.

The present invention provides a method of ameliorating joint pain in a patient, comprising injecting the above described compositions into the joint. In one embodiment, the joint is the knee.

The present invention provides a method of ameliorating the symptoms of osteoarthritis in a patient, comprising injecting the above described compositions into an afflicted joint of the patient.

The present invention also provides a method of administering a visco-supplement, comprising forming an emulsion containing the visco-supplement and injecting said emulsion into a patient in need thereof. In one embodiment, the emulsion comprises the visco-supplement, a biodegradable polymer, a solvent, and a surfactant. In one embodiment, the visco-supplement is sodium hyaluronate.

The present compositions are further described in the following examples.

### EXAMPLES

### Example 1

### Emulsions

To obtain stable and easily injectable emulsions containing aqueous solution of sodium hyaluronate, emulsions of interest were formed in two parts:

Part A: A depot vehicle containing Poly (D,L-lactide-co-glycolide) (PLGA), available as 50:50 RESOMER® RG502 (PLGA RG 502), or poly(caprolactone-glycolic acid-L, lactic acid) (PCL-GA-LA) synthesized as described in the examples 1 and 2 in co-pending U.S. Ser. No. 10/857609 (ALZO540), filed May 28, 2004, the entirety of which is incorporated herein by reference, was weighed and dispensed into a Keyence hybrid mixer bowl. Appropriate amount of solvent was weighed and added into the mixing bowl. The mixing bowl was tightly sealed, placed into the Keyence hybrid mixer (model HM-501, Keyence, Japan), and mixed for 5-10 minutes at mixing speed (revolution 2000 rpm and rotation 800 rpm). This mixture also included a surfactant, in amount of between 0.1-5% by weight; and

Part B: An aqueous solution of sodium hyaluronate (high molecular weight, MW>1000 kD or lightly crosslinked).

Parts A and B were loaded into two separated syringes connected with dual LUER-LOK™. The depot vehicle and aqueous solution of sodium hyaluronate were mixed by pushing both plungers back and forth 20-100 times to produce a homogenous emulsion. Subsequently, the emulsion could be injected through a 21-gauge 1-inch needle into a desired joint space.

### Example 2

### Preparation of Part A

Table 1 summarizes the list of depot vehicles with different solvents, and different levels of surfactant in the vehicle. An amount of biodegradable polymer, a terpolymer of lactic acid, glycolic acid, and poly ε-caprolactone (PCL-GA-LA (40/55/5)), sufficient to produce the percentage listed in TABLE 1, was dissolved in a solvent, Benzyl Benzoate (BB) or a mixture of BB and Benzyl Alcohol (BA). Surfactant PLURONIC F68 was added to the polymer solutions with amount between 0.1-2% by weight.

TABLE 1

| Vehicle | PCL-GA-LA (wt %) | BB (wt %) | BA (wt %) | F68 (wt %) |
|---|---|---|---|---|
| 1 | 40.0 | 60.0 | — | — |
| 2 | 39.2 | 58.8 | — | 2.0 |
| 3 | 40.0 | 45.0 | 15.0 | — |
| 4 | 39.2 | 44.1 | 14.7 | 2.0 |
| 5 | 39.6 | 59.4 | — | 1.0 |
| 6 | 39.8 | 59.7 | — | 0.5 |
| 7 | 39.96 | 59.94 | — | 0.1 |
| 8 | 40.0 | 60.0 | — | — |
| 9 | 39.2 | 58.8 | — | 2.0 |
| 10 | 40.0 | 45.0 | 15.0 | — |
| 11 | 39.2 | 44.1 | 14.7 | 2.0 |
| 12 | 39.6 | 59.4 | — | 1.0 |
| 13 | 39.8 | 59.7 | — | 0.5 |
| 14 | 39.96 | 59.94 | — | 0.1 |

Table 2 summarizes the list of depot vehicles with different solvents, and different levels of surfactant in the vehicle. An amount of biodegradable polymer, Poly (D,L-lactide-co-glycolide) (PLGA), available as 50:50 RESOMER® RG502 (PLGA RG 502), sufficient to produce the percentage listed in TABLE 1 was dissolved in a solvent, Benzyl Benzoate (BB) or a mixture of BB and Benzyl Alcohol (BA). Surfactant TWEEN 20 was added to the polymer solutions with amount between 0.1-2% by weight.

TABLE 2

| | PLGA RG502 | | | |
|---|---|---|---|---|
| Vehicle | (wt %) | BB (wt %) | BA (wt %) | TWEEN 20 (wt %) |
| 15 | 45.0 | 55.0 | — | — |
| 16 | 44.1 | 53.9 | — | 2.0 |
| 17 | 50.0 | 37.5 | 12.5 | — |
| 18 | 49.0 | 36.8 | 12.2 | 2.0 |

Table 3 summarizes the list of depot vehicles with different solvents, and different levels of surfactant in the vehicle. An amount of biodegradable polymer, a terpolymer of lactic acid, glycolic acid, and poly ε-caprolactone (PCL-GA-LA), sufficient to produce the percentage listed in TABLE 3 was dissolved in a solvent, Benzyl Benzoate (BB) or a mixture of BB and Benzyl Alcohol (BA). Surfactant TWEEN 80 was added to the polymer solutions with amount between 0.1-2% by weight.

TABLE 3

| | PCL-GA-LA | | | |
|---|---|---|---|---|
| Vehicle | (wt %) | BB (wt %) | BA (wt %) | TWEEN 80 (wt %) |
| 19 | 40.0 | 60.0 | — | — |
| 20 | 39.2 | 58.8 | — | 2.0 |
| 21 | 40.0 | 45.0 | 15.0 | — |
| 22 | 39.2 | 44.1 | 14.7 | 2.0 |

### Example 3

### Preparation of Aqueous Solution of Hyaluronate (Part B)

It is understood that the aqueous solution of hyaluronate (Part B) can be sourced from the commercially available products, such as ARZT®, HYALGAN®, SYNVISC®, ARTHREASE®, ORTHOVISC®, and the like.

The aqueous solution of hyaluronate (Part B) is also prepared by dissolving sodium hyaluronate (Genzyme, USA) in phosphate buffer, pH 7.4 with the sodium hyaluronate concentration of 0.5-5% by weight.

### Example 4

### Characterization of Emulsions

Using the vehicles from Example 2, about 0.5 mL of vehicle was loaded into a 3 mL syringe, and approximately 0.5 mL of sodium hyaluronate loaded into another 3 mL syringe attached together via a LUER-LOK™ connector (metal). The depot vehicle and sodium hyaluronate were mixed by pushing both plungers back and forth 100 times to produce a homogenous mixture. After mixing, the entire contents of the emulsion were added into one syringe, and a 21-guage 1 inch needle was attached. Subsequently, the mixture was injected through a 21-gauge 1-inch needle into a weighing pan for observations.

The depot vehicle (Part A) with 2 wt % surfactant formed homogenous emulsions upon mixing with the aqueous solution of hyaluronate (Part B) based on the procedure described above. Injection of this mixture through a 21G 1″ needle produced smooth, creamy, and homogeneous emulsions. Moreover, this mixture was easily injectable.

In contrast, vehicles with no surfactant, showed unevenly distributed droplets of polymer in mixtures, and resulted in poor injectability.

As long as the surfactant, PLURONIC F68, content in the depot vehicles was equal or higher than 0.5 wt %, smooth, creamy, and homogeneous emulsions were produced.

Ratios of Part A to Part B in the range of 2:1-1:2 formed homogeneous emulsions as long as surfactant was present.

A set of emulsions were prepared and stored at 4° C. for stability study. After four days of storage at refrigerated temperature (2-8° C.), homogenous emulsions showed no obvious phase separation.

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

What is claimed:

1. A composition comprising:
a biodegradable polymer;
a solvent;
hyaluronic acid or a salt thereof; and
a surfactant.

2. The composition of claim 1, wherein the biodegradable polymer and the solvent comprise a depot vehicle.

3. The composition of claim 2, wherein the depot vehicle further comprises the surfactant.

4. The composition of claim 1, wherein the hyaluronic acid or a salt thereof is:
a) in a dry form and is about 0.1-50 wt. % of the composition; or
b) in an aqueous solution, wherein the aqueous solution is about 30-50 wt. % of the composition.

5. The composition of claim 1, wherein the biodegradable polymer is selected from the group consisting of polylactides, polyglycolides, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), and poly(amino acids), and copolymers, terpolymers and mixtures thereof.

6. The composition of claim 5, wherein the biodegradable polymer is a lactic acid-containing polymer.

7. The composition of claim 6, wherein the lactic acid is present in a range from about 45 wt. % to about 99 wt. % of the polymer.

8. The composition of claim 6, further comprising glycolic acid present in a range from about 35 wt. % to about 65 wt. % of the polymer.

9. The composition of claim 1, wherein the biodegradable polymer is a terpolymer of lactic acid, glycolic acid, and poly ε-caprolactone.

10. The composition of claim 9, wherein the biodegradable polymer is a terpolymer of 5 wt % lactic acid, 55 wt % glycolic acid, and 40 wt % poly ε-caprolactone.

11. The composition of claim 1, wherein the biodegradable polymer is present in a range from about 15 wt % to about 60 wt % of the composition.

12. The composition of claim 1, wherein the solvent is selected from the group consisting of aromatic alcohols, lower alkyl esters of aryl acids, lower aralkyl esters of aryl acids, aryl ketones, aralkyl ketones, lower alkyl ketones, lower alkyl esters of citric acid, and combinations thereof.

13. The composition of claim 1, wherein the solvent is ethyl oleate, benzyl benzoate, ethyl benzoate, lauryl lactate, benzyl alcohol, lauryl alcohol, glycofurol, ethanol, tocopherol, polyethylene glycol, triacetin, a triglyceride, an alkyltriglyceride, a diglyceride, sesame oil, peanut oil, castor oil, olive oil, cottonseed oil, perfluorocarbon, N-methyl-pyrrolidone, DMSO, glycerol, oleic acid, glycofurol, lauryl lactate, perfluorocarbon, propylene carbonate, or mixtures thereof.

14. The composition of claim 1, wherein the solvent is methyl benzoate, ethyl benzoate, n-propyl benzoate, isopropyl benzoate, butyl benzoate, isobutyl benzoate, sec-butyl benzoate, tert-butyl benzoate, isoamyl benzoate, or benzyl benzoate.

15. The composition of claim 1, wherein the solvent is benzyl benzoate, benzyl alcohol, or benzyl benzoate and benzyl alcohol.

16. The composition of claim 1, wherein the solvent is present in a range from about 15 wt % to about 60 wt % of the composition.

17. The composition of claim 1, wherein the surfactant is an ionic surfactant, nonionic surfactant, or a polymeric surfactant.

18. The composition of claim 1, wherein the surfactant is a polyoxyethylene sorbitan-containing composition, a block copolymer of propylene oxide and ethylene oxide, a block copolymer derived from the addition of ethylene oxide and propylene oxide to ethylenediamine, polyethelene glycol, or polyethylene oxide.

19. The composition of claim 1, wherein the surfactant is polyoxyethylene sorbitan monolaureate, polyoxyethylene sorbitan monooleat, or a block copolymer of propylene oxide and ethylene oxide is of a formula HO-(ethylene oxide)ₓ-(propylene oxide)_{y}-(ethylene oxide)_{x′}-H, wherein x is about 79, y is about 28, and x′ is about 79.

20. The composition of claim 1, wherein the surfactant is present in a range from about 0.01 wt % to about 5 wt % of the composition.

21. The composition of claim 1, wherein the surfactant is present in a range from about 0.04 wt. % to about 2.0 wt. % of the composition.

22. An injectable depot emulsion composition for the sustained delivery of hyaluronic acid or a salt thereof comprising the composition of claim 1.

23. A method of ameliorating joint pain in a patient, comprising:
injecting the composition of claim 1 into the joint.

24. A method of ameliorating the symptoms of osteoarthritis in a patient, comprising:
injecting the composition of claim 1 into an afflicted joint of the patient.

25. A method of administering a visco-supplement, comprising:
forming an emulsion containing the visco-supplement; and
injecting said emulsion into a patient in need thereof.

26. The method of claim 25, wherein the emulsion comprises the visco-supplement, a biodegradable polymer, a solvent, and a surfactant.
